(19) United States
(12) Patent Application Publication
(10) Pub. No.: US 2021/0010073 A1
(43) Pub. Date: Jan. 14, 2021

(54) PREPARATION OF NUCLEIC ACID LIBRARIES FROM RNA AND DNA

(71) Applicant: Illumina, Inc., San Diego, CA (US)

(72) Inventors: Hongxia Xu, San Francisco, CA (US); Dan Cao, Mountain View, CA (US); Alex Aravanis, San Mateo, CA (US)

(21) Appl. No.: 16/609,869

(22) PCT Filed: Mar. 20, 2019
(86) PCT No.: PCT/US2019/023114

§ 371 Date: Oct. 31, 2019

Related U.S. Application Data
(60) Provisional application No. 62/646,487, filed on Mar. 22, 2018.

(51) Int. Cl. C12Q 1/6869; C12Q 1/6806; C12N 9/12; C40B 40/08

(52) U.S. Cl. CPC C12Q 1/6869; C12Q 1/6806; C12Q 2531/113; C40B 40/08; C12N 9/1276

(57) ABSTRACT

Some embodiments of the methods and compositions provided herein relate to the preparation and use of nucleic acid libraries derived from RNA and DNA. In some embodiments, a nucleic acid library can be prepared by tagging polynucleotides derived from RNA. Some embodiments include the analysis of sequence data from such libraries.

### CROSS-REFERENCE TO RELATED APPLICATIONS

This application claims priority to U.S. Prov. App. No. 62/646,487 filed Mar. 22, 2018 entitled “PREPARATION OF NUCLEIC ACID LIBRARIES FROM RNA AND DNA” which is incorporated by reference herein in its entirety.

### FIELD OF THE INVENTION

Some embodiments of the methods and compositions provided herein relate to the preparation and use of nucleic acid libraries derived from RNA and DNA. In some embodiments, a nucleic acid library can be prepared by tagging polynucleotides derived from RNA.

### BACKGROUND OF THE INVENTION

Whole genome sequencing, genotyping, targeted resequencing, and gene expression analyses of tissue samples can be of significant importance for identifying disease biomarkers, accurately diagnosing and prognosticating diseases, and selecting the proper treatment for a patient. For example, nucleic acid sequence analysis of tumor tissue excised from a patient can be used to determine the presence or absence of particular genetic biomarkers, such as somatic variants, structural rearrangements, point mutations, deletions, insertions, and/or the presence or absence of particular genes. Cell-free samples can be used to prepare nucleic acid libraries for sequence analysis. However, nucleic acids that include disease biomarkers in such libraries can be rare and difficult to detect. Therefore, there is a desire for increased sensitivity in the detection of disease biomarkers.

### SUMMARY OF THE INVENTION

Some embodiments include a method for preparing a library of nucleic acids comprising: (a) hybridizing a plurality of polynucleotides with a plurality of primers comprising tags, wherein the plurality of polynucleotides comprises RNA and DNA; (b) extending the hybridized primers with a reverse transcriptase; and (c) generating a library of nucleic acids from the extended primers and the DNA. Some embodiments also include (d) sequencing the library of nucleic acids. Some embodiments also include (e) identifying polynucleotide sequences comprising the tags, thereby identifying sequences derived from the RNA polynucleotides of the plurality of polynucleotides. Some embodiments also include identifying polynucleotide sequences lacking the tags, thereby identifying sequences derived from the DNA polynucleotides of the plurality of polynucleotides.

In some embodiments, the plurality of primers comprises different sequences. In some embodiments, each primer comprises a different sequence. In some embodiments, the plurality of primers comprises greater than 10,000 different sequences. In some embodiments, the plurality of primers comprises greater than 100,000 different sequences. In some embodiments, the plurality of primers comprises random hexamer sequences. In some embodiments, the plurality of primers comprises the same tag.

In some embodiments, the reverse transcriptase lacks a DNA-dependent polymerase activity. In some embodiments, the reverse transcriptase is selected from the group consisting of avian myeloblastosis virus (AMV) reverse transcriptase, moloney murine leukemia virus (MMLV) reverse transcriptase, human immunovirus (HIV) reverse transcriptase, equine infectious anemia virus (EIAV) reverse transcriptase, Rous-associated virus-2 (RAV2) reverse transcriptase, C. hydrogenoformans DNA polymerase, T. thermus DNA polymerase, T. flavus DNA polymerase, and functional variants thereof.

In some embodiments, (b) is performed in the presence of the DNA polynucleotides. In some embodiments, (b) comprises generating double-stranded cDNA from the extended primers. In some embodiments, (c) comprises contacting the extended primers and DNA polynucleotides with a reagent selected from the group consisting of a kinase, a ligase, a transposon, a polymerase, and a sequencing adaptor.

In some embodiments, the plurality of polynucleotides is cell-free. In some embodiments, the plurality of polynucleotides is obtained from a sample selected from the group consisting of serum, interstitial fluid, lymph, cerebrospinal fluid, sputum, urine, milk, sweat, and tears.

Some embodiments include a method for preparing a library of nucleic acids comprising: (a) hybridizing a plurality of polynucleotides with a plurality of primers, wherein the plurality of polynucleotides comprises RNA and DNA; (b) extending the hybridized primers with a reverse transcriptase; and (c) generating a library of nucleic acids from the extended primers and the DNA.

In some embodiments, the plurality of polynucleotides is cell-free. In some embodiments, the plurality of polynucleotides is obtained from a sample selected from the group consisting of serum, interstitial fluid, lymph, cerebrospinal fluid, sputum, urine, milk, sweat, and tears.

In some embodiments, the plurality of primers comprises different sequences. In some embodiments, each primer comprises a different sequence. In some embodiments, the plurality of primers comprises greater than 10,000 different sequences. In some embodiments, the plurality of primers comprises greater than 100,000 different sequences. In some embodiments, the plurality of primers comprises random hexamer sequences.

In some embodiments, the reverse transcriptase lacks a DNA-dependent polymerase activity. In some embodiments, the reverse transcriptase is selected from the group consisting of avian myeloblastosis virus (AMV) reverse transcriptase, moloney murine leukemia virus (MMLV) reverse transcriptase, human immunovirus (HIV) reverse transcriptase, equine infectious anemia virus (EIAV) reverse transcriptase, Rous-associated virus-2 (RAV2) reverse transcriptase, C. hydrogenoformans DNA polymerase, T. thermus DNA polymerase, T. flavus DNA polymerase, and functional variants thereof.

In some embodiments, (b) is performed in the presence of the DNA polynucleotides. In some embodiments, (b) comprises generating double-stranded cDNA from the extended primers. In some embodiments, (c) comprises contacting the extended primers and DNA polynucleotides with a reagent selected from the group consisting of a kinase, a ligase, a transposon, a polymerase, and a sequencing adaptor.

Some embodiments include a method of identifying a nucleic acid in a sample of nucleic acids, comprising: (i) obtaining sequence data from a library of nucleic acids prepared from a sample of nucleic acids by any one of the foregoing methods; and (ii) identifying a polynucleotide sequence comprising a tag, thereby identifying a sequence derived from a RNA polynucleotide of the plurality of polynucleotides. Some embodiments also include (iii) identifying a variant in the polynucleotide sequence comprising a tag. In some embodiments, the variant is selected from the group consisting of a single nucleotide polymorphism (SNP), a deletion, an insertion, a substitution, a translocation, a duplication, and a gene fusion. Some embodiments also include identifying a reverse transcription error in the polynucleotide sequence comprising a tag. Some embodiments also include comparing the polynucleotide sequence comprising a tag with a reference sequence. In some embodiments, the reference sequence is derived from a DNA polynucleotide of the library of nucleic acids. In some embodiments, the sample comprises cell-free nucleic acids. In some embodiments, the RNA polynucleotide is an RNA selected form the group consisting of mRNA, tRNA, ribosomal RNA, non-coding RNA, piRNA, siRNA, lncRNA, snRNA, snRNA, miRNA, snoRNA, viral RNA, bacterial RNA, and a ribozyme.

Some embodiments also include a kit for preparing a library of nucleic acids comprising: a reverse transcriptase; and a plurality of primers comprising tags, wherein each primer is different. In some embodiments, the plurality of primers comprises the same tag. Some embodiments also include a component selected from the group consisting of a kinase, an RNase, a ligase, a transposon, a polymerase, and a sequencing adaptor. In some embodiments, the reverse transcriptase lacks DNA-dependent polymerase activity. In some embodiments, the reverse transcriptase is selected from the group consisting of avian myeloblastosis virus (AMV) reverse transcriptase, moloney murine leukemia virus (MMLV) reverse transcriptase, human immunovirus (HIV) reverse transcriptase, equine infectious anemia virus (EIAV) reverse transcriptase, Rous-associated virus-2 (RAV2) reverse transcriptase, C. hydrogenoformans DNA polymerase, T. thermus DNA polymerase, T. flavus DNA polymerase, and functional variants thereof.

### BRIEF DESCRIPTION OF THE DRAWINGS

FIG. 1 is a schematic view of an embodiment for preparing a nucleic acid library from RNA and DNA, and sequencing the same.

FIG. 2 is a graph of the concentration of certain nucleic acids in samples from various patients.

FIG. 3 is a graph of the number of certain sequences obtained from either a library prepared by a method with (RT counts) or without (mock RT counts) a reverse transcription step.

FIG. 4 is a graph of the ratio of coverage for libraries prepared by a method with a reverse transcription step (RT) vs. a method without a reverse transcription step (mock RT), for certain gene regions tested in a NSCLC V1 panel.

FIG. 5 is a graph of the number of mutations that were found with an increased frequency in the library prepared with a reverse transcription step.

FIG. 6 is a graph of the number of reads from a library prepared with tagged random hexamers either with reverse transcriptase (A); or without reverse transcriptase (B).

### DETAILED DESCRIPTION

Embodiments of the methods and compositions provided herein relate to the preparation and use of nucleic acid libraries derived from RNA and DNA. In some embodiments, a nucleic acid library can be prepared by tagging polynucleotides derived from RNA.

Bodily fluids, such as serum, tears, urine, and sweat contain cell-free nucleic acids. Such nucleic acids can include disease biomarkers. However, the frequency or concentration of such biomarkers in those fluids can be extremely low. Some embodiments include preparing nucleic acid libraries from RNA and DNA which increase the sensitivity of detecting certain nucleic acids, including disease biomarkers.

Some embodiments include preparing a library of nucleic acids by reverse transcribing RNA with a primer that includes a tag and incorporates the sequence of the tag into polynucleotides derived from the RNA. Thus, a tag can identify a sequence that is derived from the RNA. In some embodiments, distinguishing the source of a nucleic acid sequence can be useful to determine whether a variant could be the result of library preparation, such as a reverse transcription step. In some embodiments, distinguishing the source of a nucleic acid sequence can be useful to identify splice variants, tissue-specific variants, non-coding RNAs, and certain gene-fusions. Non-coding RNA, such as long non-coding RNA (lncRNA) can be useful to identify and characterize certain cancer types. See e.g., Yan, X., et al., (2015) “Comprehensive Genomic Characterization of Long Non-coding RNAs across Human Cancers”, Cancer Cell 28:529-540 which is incorporated by reference in its entirety. Cell-free lncRNA may be more stable in plasma than other RNAs, such as protein coding RNA due to secondary structure.

As used herein, “polynucleotide” can refer to a polymeric form of nucleotides of any length, including deoxyribonucleotides and/or ribonucleotides, or analogs thereof. Polynucleotides can have any three-dimensional structure and may perform any function, known or unknown. The structure of a polynucleotide can also be referenced to by its 5′ or 3′ end or terminus, which indicates the directionality of the polynucleotide. Adjacent nucleotides in a single-strand of polynucleotides are typically joined by a phosphodiester bond between their 3′ and 5′ carbons. However, different internucleotide linkages could also be used, such as linkages that include a methylene, phosphoramidate linkages, etc. This means that the respective 5′ and 3′ carbons can be exposed at either end of the polynucleotide, which may be called the 5′ and 3′ ends or termini. The 5′ and 3′ ends can also be called the phosphoryl (PO₄) and hydroxyl (OH) ends, respectively, because of the chemical groups attached to those ends. The term polynucleotide also refers to both double and single-stranded molecules. Examples of polynucleotides include a gene or gene fragment, genomic DNA, genomic DNA fragment, exon, intron, messenger RNA (mRNA), transfer RNA, ribosomal RNA, non-coding RNA (ncRNA) such as PIWI-interacting RNA (piRNA), small interfering RNA (siRNA), and long non-coding RNA (lncRNA), small hairpin (snRNA), small nuclear RNA (snRNA), micro RNA (miRNA), small nucleolar RNA (snoRNA) and viral RNA, ribozyme, cDNA, recombinant polynucleotide, branched polynucleotide, plasmid, vector, isolated DNA of any sequence, isolated RNA of any sequence, nucleic acid probe, primer or amplified copy of any of the foregoing. A polynucleotide can include modified nucleotides, such as methylated nucleotides and nucleotide analogs including nucleotides with non-natural bases, nucleotides with modified natural bases such as aza- or deaza-purines. A polynucleotide can be composed of a specific sequence of four nucleotide bases: adenine (A); cytosine (C); guanine (G); and thymine (T). Uracil (U) can also be present, for example, as a natural replacement for thymine when the polynucleotide is RNA. Uracil can also be used in DNA. Thus, the term ‘sequence’ refers to the alphabetical representation of a polynucleotide or any nucleic acid molecule, including natural and non-natural bases.

As used herein, “RNA molecule” or ribonucleic acid molecule can refer to a polynucleotide having a ribose sugar rather than deoxyribose sugar and typically uracil rather than thymine as one of the pyrimidine bases. An RNA molecule is generally single-stranded, but can also be double-stranded. In the context of an RNA molecule from an RNA sample, the RNA molecule can include the single-stranded molecules transcribed from DNA in the cell nucleus, mitochondrion, chloroplast or bacterial cell, which have a linear sequence of nucleotide bases that is complementary to the DNA strand from which it is transcribed.

As used herein, “hybridization”, “hybridizing” or grammatical equivalent thereof, can refer to a reaction in which one or more polynucleotides react to form a complex that is formed at least in part via hydrogen bonding between the bases of the nucleotide residues. The hydrogen bonding can occur by Watson-Crick base pairing, Hoogstein binding, or in any other sequence-specific manner. The complex can have two strands forming a duplex structure, three or more strands forming a multi-stranded complex, a single self-hybridizing strand, or any combination of thereof. The strands can also be cross-linked or otherwise joined by forces in addition to hydrogen bonding.

As used herein, “extending”, “extension” or any grammatical equivalents thereof can refer to the addition of dNTPs to a primer, polynucleotide or other nucleic acid molecule by an extension enzyme such as a polymerase. For example, in some methods disclosed herein, the resulting extended primer includes sequence information of an RNA. While some embodiments are discussed as performing extension using a polymerase such as a DNA polymerase, or a reverse transcriptase, extension can be performed in any other manner well known in the art. For example, extension can be performed by ligating short pieces of random oligonucleotides together, such as oligonucleotides that have hybridized to a strand of interest.

As used herein, “reverse transcription” can refer to the process of copying the nucleotide sequence of a RNA molecule into a DNA molecule. Reverse transcription can be done by contacting an RNA template with a RNA-dependent DNA polymerase, also known as a reverse transcriptase. A reverse transcriptase is a DNA polymerase that transcribes single-stranded RNA into single-stranded DNA. Depending on the polymerase used, the reverse transcriptase can also have RNase H activity for subsequent degradation of the RNA template.

As used herein, “complementary DNA” or “cDNA” can refer to a synthetic DNA reverse transcribed from RNA through the action of a reverse transcriptase. The cDNA may be single-stranded or double-stranded and can include strands that have either or both of a sequence that is substantially identical to a part of the RNA sequence or a complement to a part of the RNA sequence.

As used herein, “cDNA library” can refer to a collection of DNA sequences generated from RNA sequences. The cDNA library can represent the RNA present in the original sample from which the RNA was extracted. In some embodiments, the cDNA library can represent the RNA present in a cell-free sample of nucleic acids. In some embodiments, a cDNA library can represent all or a part of a transcriptome of a given cell or population of cells including messenger RNA (mRNA), ribosomal RNA (rRNA), transfer RNA (tRNA) and other non-coding RNA (ncRNA) produced in one cell or a population of cells.

As used herein, “ligation” or “ligating” or other grammatical equivalents thereof can refer to the joining of two nucleotide strands by a phosphodiester bond. Such a reaction can be catalyzed by a ligase. A ligase refers to a class of enzymes that catalyzes this reaction with the hydrolysis of ATP or a similar triphosphate.

As used herein, “derived” when used in reference to a sequence of a nucleic acid can refer to the source from which the nucleic acid was obtained. For example, a sequence can be obtained from a nucleic acid that was derived from an RNA molecule in a sample. A nucleic acid molecule that is derived from a particular source or origin can nonetheless be subsequently copied or amplified. The sequence of the resulting copies or amplicons can be referred to as having been derived from the source or origin.

### Preparing Nucleic Acids Libraries

Some embodiments include methods of preparing a library of nucleic acids. Some such embodiments can include obtaining a sample that includes a plurality of polynucleotides comprising RNA and DNA; hybridizing the plurality of polynucleotides with a plurality of primers; and extending the hybridized primers with a reverse transcriptase. In some such embodiments, the primers comprise tags. Some embodiments also include generating a library of nucleic acids from the extended primers and the DNA.

In some embodiments, a sample can include cell-free nucleic acids, such as RNA and DNA. As used herein, “cell-free” in reference to a nucleic acid can refer to a nucleic acid which is removed from a cell in vivo. The removal of the nucleic acid can be a natural process such as necrosis or apoptosis. Cell-free nucleic acids can be obtained from blood, or a fraction thereof, such as serum. Cell-free nucleic acids can be obtained from other bodily fluids or tissues, examples include interstitial fluid, lymph, cerebrospinal fluid, sputum, urine, milk, sweat, and tears.

Some embodiments include the use of primers. As used herein, “primer” can refer to a short polynucleotide, generally with a free 3′-OH group, that binds to a target or template polynucleotide present in a sample by hybridizing with the target or template, and thereafter promoting extension of the primer to form a polynucleotide complementary to the target or template. Primers can include polynucleotides ranging from 5 to 1000 or more nucleotides. In some embodiments, the primer has a length of at least 4 nucleotides, 5 nucleotides, 10 nucleotides, 15 nucleotides, 20 nucleotides, 25 nucleotides, 30 nucleotides, 35 nucleotides, 40 nucleotides, 45 nucleotides, 50 nucleotides, 60 nucleotides, 70 nucleotides, 80 nucleotides, 90 nucleotides, 100 nucleotides, or a length within a range of any two of the foregoing lengths.

Primers can include a random nucleotide sequence. As used herein, “random nucleotide sequence” can refer to a varied sequence of nucleotides that when combined with other random nucleotide sequences in a population of polynucleotides represent all or substantially all possible combinations of nucleotides for a given length of nucleotides. For example, because of the four possible nucleotides present at any given position, a sequence of two random nucleotides in length has 16 possible combinations, a sequence of three random nucleotides in length has 64 possible combinations, or a sequence of four random nucleotides in length has 265 possible combinations. A random nucleotide sequence has the potential to hybridize to any target polynucleotide in a sample. A random sequence in a primer can include several consecutive nucleotides and have a length of at least 4 nucleotides, 5 nucleotides, 10 nucleotides, 15 nucleotides, 20 nucleotides, 25 nucleotides, 30 nucleotides, 35 nucleotides, 40 nucleotides, 45 nucleotides, 50 nucleotides, 60 nucleotides, 70 nucleotides, 80 nucleotides, 90 nucleotides, 100 nucleotides, or a length within a range of any two of the foregoing lengths. In some embodiments, a plurality of primers can include primers that include different random sequences. Some embodiments include the use of a plurality of primers. In some embodiments, each primer comprises a different sequence. In some embodiments, a plurality of primers can include at least 1000, 10,000, 100,000, 1,000,000, 10,000,000, 100,000,000 different sequences, or a number of different sequences in a range between any two of the foregoing numbers.

Primers can include tags. As used herein, “tag” can refer to a nucleotide sequence that is attached to a primer or probe, or incorporated into a polynucleotide, that allows for the identification, tracking, or isolation of the attached primer, probe or polynucleotide in a subsequent reaction or step in a method or process. The nucleotide composition of a tag can also be selected so as to allow hybridization to a complementary probe, such as a probe on a solid support, such as the surface of an array, or hybridization to a complementary primer used to selectively amplify a target sequence. A tag can include several consecutive nucleotides and have a length of at least 3 nucleotides, 4 nucleotides, 5 nucleotides, 10 nucleotides, 15 nucleotides, 20 nucleotides, 25 nucleotides, 30 nucleotides, 35 nucleotides, 40 nucleotides, 45 nucleotides, 50 nucleotides, or a length within a range of any two of the foregoing lengths. A tag can be a sequence at the 5′ end of a primer, at the 3′ end of a primer, or can be a sequence within a primer. In some embodiments, a tag is a sequence at the 3′ end of a primer. In some embodiments, a plurality of primers can each have different tags. In some embodiments, a plurality of primers can each have the same tag.

Some embodiments include the use of a reverse transcriptase. Reverse transcriptases include RNA-dependent DNA polymerases. Examples of reverse transcriptases include avian myeloblastosis virus (AMV) reverse transcriptase, moloney murine leukemia virus (MMLV) reverse transcriptase, human immunovirus (HIV) reverse transcriptase, equine infectious anemia virus (EIAV) reverse transcriptase, Rous-associated virus-2 (RAV2) reverse transcriptase, C. hydrogenoformans DNA polymerase, T. thermus DNA polymerase, T. flavus DNA polymerase, and functional variants thereof. In some embodiments, the reverse transcriptase can lack a DNA-dependent polymerase activity. In some embodiments, a reverse transcriptase can extend primers hybridized to RNA in the presence or absence of DNA. Extension of a primer hybridized to an RNA generates a single-stranded cDNA. As such, a cDNA library can be generated from the RNA in sample of nucleic acids. Some embodiments also include the generation of double-stranded cDNA from the extended primers using a DNA-dependent DNA polymerase and nucleotides.

Some embodiments include generating a library of nucleic acids from target nucleic acids comprising the extended primers comprising tags. In some such embodiments, target nucleic acids can also include the extended primers comprising tags and DNA, such as cell-free DNA. An example method to generate a library of nucleic acids from target nucleic acids includes tagmentation. As used herein, “tagmentation” can refer to the insertion of transposons into target nucleic acids such that the transposon cleaves the target nucleic acids, and adds adaptor sequences to the ends of the cleaved target nucleic acids. Example methods of tagmentation are disclosed in U.S. Pat. Nos. 9,115,396; 9,080,211; 9,040,256; U.S. patent application publication 2014/0194324, each of which is incorporated herein by reference in its entirety. Another example method includes the ligation of adaptor sequences to the ends of target nucleic acids with a ligase. Ligation-based library preparation methods often make use of an adaptor design which can incorporate sequencing primer site, amplification primer site, and/or an index sequence at the initial ligation step and often can be used to prepare samples for single-read sequencing, paired-end sequencing and multiplexed sequencing. For example, target nucleic acids may be end repaired by a fill-in reaction, an exonuclease reaction or a combination thereof. In some embodiments the resulting blunt-end repaired nucleic acid can then be extended by a single nucleotide, which is complementary to a single nucleotide overhang on the 3′ end of an adapter/primer. Any nucleotide can be used for the extension/overhang nucleotides. In some embodiments nucleic acid library preparation comprises ligating an adapter oligonucleotide. Adapter oligonucleotides are often complementary to flow-cell anchors, and sometimes are utilized to immobilize a nucleic acid library to a solid support. In some embodiments, an adapter oligonucleotide comprises an identifier, one or more sequencing primer hybridization sites such as sequences complementary to universal sequencing primers, single end sequencing primers, paired end sequencing primers, multiplexed sequencing primers, and the like, or combinations thereof such as adapter/sequencing, adapter/identifier, adapter/identifier/sequencing.

In some embodiments, a nucleic acid library or parts thereof can be amplified using amplification primer sites in adaptor sequences. Nucleic acid libraries can be amplified by PCR-based methods, or isothermal amplification methods. Examples of different types of amplification methods include multiplex PCR, digital PCR (dPCR), dial-out PCR, allele-specific PCR, asymmetric PCR, helicase-dependent amplification, hot start PCR, ligation-mediated PCR, miniprimer PCR, multiplex ligation-dependent probe amplification (MLPA), nested PCR, quantitative PCR (qPCR), reverse transcription PCR (RT-PCR), solid phase PCR, ligase chain reaction, strand displacement amplification (SDA), transcription mediated amplification (TMA) and nucleic acid sequence based amplification (NASBA), as described in U.S. Pat. No. 8,003,354 which is incorporated by reference in its entirety. In some embodiments, amplification can occur with amplification primers attached a solid phase. Formats that utilize two species of primer attached to the surface are often referred to as bridge amplification because double-stranded amplicons form a bridge-like structure between the two surface-attached primers that flank the template sequence that has been copied. Example reagents and conditions that can be used for bridge amplification are described in U.S. Pat. No. 5,641,658; U.S. Patent Publ. No. 2002/0055100; U.S. Pat. No. 7,115,400; U.S. Patent Publ. No. 2004/0096853; U.S. Patent Publ. No. 2004/0002090; U.S. Patent Publ. No. 2007/0128624; and U.S. Patent Publ. No. 2008/0009420, each of which is incorporated herein by reference. Other methods for amplification of nucleic acids can include oligonucleotide extension and ligation, rolling circle amplification (RCA) and oligonucleotide ligation assay (OLA). See e.g., U.S. Pat. Nos. 7,582,420, 5,185,243, 5,679,524 and 5,573,907 each of which is incorporated herein by reference in its entirety. Examples of primer extension and ligation primers that can be specifically designed to amplify a nucleic acid of interest are disclosed in U.S. Pat. Nos. 7,582,420 and 7,611,869 each of which is incorporated herein by reference in its entirety. Example isothermal amplification methods include multiple displacement amplification (MDA) which is disclosed in Dean et al., Proc. Natl. Acad. Sci. USA 99:5261-66 (2002); isothermal strand displacement nucleic acid amplification disclosed in U.S. Pat. No. 6,214,587, each of the foregoing references is incorporated herein by reference in its entirety. Additional description of amplification reactions, conditions and components are set forth in detail in the disclosure of U.S. Pat. No. 7,670,810, which is incorporated herein by reference in its entirety.

Some embodiments can include sequencing a nucleic acid. Examples of sequencing technologies include sequencing-by-synthesis (SBS). In SBS, extension of a nucleic acid primer along a nucleic acid template is monitored to determine the sequence of nucleotides in the template. The underlying chemical process can be polymerization. In a particular polymerase-based SBS embodiment, fluorescently labeled nucleotides are added to extend a primer in a template dependent fashion such that detection of the order and type of nucleotides added to the primer can be used to determine the sequence of the template. One or more amplified nucleic acids can be subjected to an SBS or other detection technique that involves repeated delivery of reagents in cycles. For example, to initiate a first SBS cycle, one or more labeled nucleotides, DNA polymerase, etc., can be flowed into/through a hydrogel bead that houses one or more amplified nucleic acid molecules. Those sites where primer extension causes a labeled nucleotide to be incorporated can be detected. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the flow cell before or after detection occurs. Washes can be carried out between the various delivery steps. The cycle can then be repeated n times to extend the primer by n nucleotides, thereby detecting a sequence of length n.

Some SBS embodiments include detection of a proton released upon incorporation of a nucleotide into an extension product. For example, sequencing based on detection of released protons can use an electrical detector and associated techniques that are commercially available. Examples of such sequencing systems are pyrosequencing such as a commercially available platform from 454 Life Sciences a subsidiary of Roche; sequencing using γ-phosphate-labeled nucleotides, such as a commercially available platform from Pacific Biosciences; and sequencing using proton detection, such as a commercially available platform from Ion Torrent subsidiary of Life Technologies.

Pyrosequencing detects the release of inorganic pyrophosphate (PPi) as particular nucleotides are incorporated into a nascent nucleic acid strand. In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system. Excitation radiation sources used for fluorescence based detection systems are not necessary for pyrosequencing procedures.

Some embodiments can utilize methods involving the real-time monitoring of DNA polymerase activity. For example, nucleotide incorporations can be detected through fluorescence resonance energy transfer (FRET) interactions between a fluorophore-bearing polymerase and γ-phosphate-labeled nucleotides, or with zero mode waveguides (ZMWs). Another useful sequencing technique is nanopore sequencing. In some nanopore embodiments, the target nucleic acid or individual nucleotides removed from a target nucleic acid pass through a nanopore. As the nucleic acid or nucleotide passes through the nanopore, each nucleotide type can be identified by measuring fluctuations in the electrical conductance of the pore.

Embodiments can include the isolation, amplification, and sequencing, of nucleic acids using various reagents. Such reagents may include, for example, lysozyme; proteinase K; random hexamers; polymerase such as Φ29 DNA polymerase, Taq polymerase, Bsu polymerase; transposase such as Tn5; primers such as P5 and P7 adaptor sequences; ligase; deoxynucleotide triphosphates; buffers; or divalent cations such as magnesium cations. Adaptors can include sequencing primer sites, amplification primer sites, and indexes. As used herein an “index” can include a sequence of nucleotides that can be used as a molecular identifier and/or barcode to tag a nucleic acid, and/or to identify the source of a nucleic acid. In some embodiments, an index can be used to identify a single nucleic acid, or a subpopulation of nucleic acids.

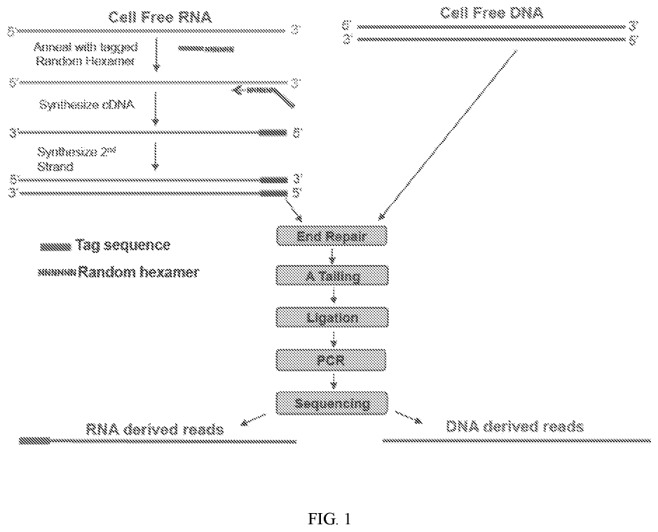

FIG. 1 depicts an example embodiment of a method of preparing a library of nucleic acids. As shown in FIG. 1, a sample comprising cell-free RNA and cell-free DNA is provided. Primers comprising random hexamer sequences and tag sequences are hybridized to the RNA. The hybridized primers are extended to generate a first cDNA strand using a reverse transcriptase. A second cDNA strand can be synthesized from the first cDNA strand to generate a double-stranded cDNA. The foregoing steps can be performed in the presence of the cell-free DNA. A library of nucleic acids can be generated from the double-stranded cDNA and cell-free DNA. Steps can include end-repair of nucleic acid molecules, A-tailing of nucleic acid molecules, ligation of adaptors, amplification of the library by PCR, and sequencing of the library. Sequences derived from the cell-free RNA can be identified by the inclusion of a tag sequence. Sequences derived from the cell-free DNA can be identified by the lack of a tag sequence.

Some embodiments include identifying a nucleic acid in a sample of nucleic acids. Some such embodiments can include obtaining sequence data from a library of nucleic acids prepared from a sample of nucleic acids by a method provided herein, and identifying a polynucleotide sequence comprising a tag, thus identifying a sequence derived from a RNA polynucleotide. Some embodiments can also include identifying a variant in the polynucleotide sequence comprising a tag. Examples of variants include a single nucleotide polymorphism (SNP), a deletion, an insertion, a substitution, a translocation, a duplication, and a gene fusion. Some embodiments also include identifying a reverse transcription error in the polynucleotide sequence comprising a tag. For example, a reverse transcriptase can introduce errors into a cDNA. Thus, identification of the source of a sequence can be useful to determine whether a variant could be the result of reverse transcription. In some embodiments, a polynucleotide sequence derived from an RNA can be compared with a reference sequence, such as the sequence of a DNA polynucleotide of the library of nucleic acids.

### Kits

Some embodiments provided herein include kits. A kit can include a reagent for preparing a nucleic acid library from a sample comprising RNA. Such kits can include a reverse transcriptase, and a plurality of primers comprising tags. Kits can also include a reagent to generate double-stranded cDNA, such as a DNA polymerase and nucleotides. Kits can also include reagents such a kinase, an RNase, a ligase, a transposon, a polymerase, and a sequencing adaptor.

### Examples

### Example 1—RNA/DNA Molecules in Serum

Droplet digital PCR (ddPCR) was used to measure the concentration of nucleic acids encoding phosphatidylinositol-4, 5-bisphosphate 3-kinase catalytic subunit alpha (PIK3CA) and B-Raf (BRAF) in serum from cancer patients and control subjects. Prior to amplification, nucleic acids were prepared with and without a reverse transcription step to provide samples containing either DNA, or DNA and reverse transcribed RNA (cDNA). For PIK3CA analysis, a 79 nt amplicon of Exon 20 of PIK3CA (dHsaCP2506262) and labeled with FAM was used (BIO-RAD, Hercules, Calif.). For BRAF analysis, a 66 nt exonic amplicon of BRAF (dHsaCP2500366) labelled with HEX was used (BIO-RAD, Hercules, Calif.).

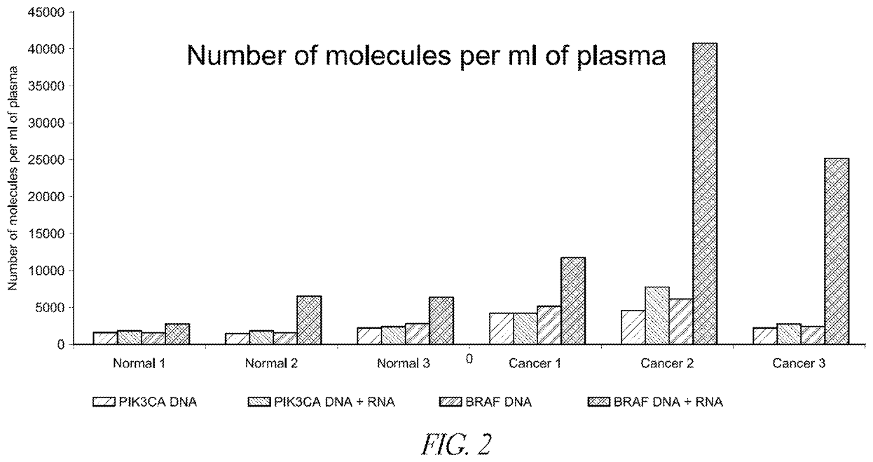

The initial serum concentrations were determined for the number of DNA molecules encoding PIK3CA and BRAF exons, and the number of DNA and RNA molecules together encoding PIK3CA and BRAF exons. FIG. 2 is a graph of the concentration of nucleic acids encoding PIK3CA and BRAF in serum from cancer patients (cancer 1, 2, and 3) and control subjects (normal 1, 2 and 3). Nucleic acid samples that had been treated with a reverse transcription step to calculate the initial concentration of exons are labeled as “DNA+RNA”. Nucleic acid samples that had not been treated with a reverse transcription step to calculate the initial concentration of exons are labeled as “DNA”.

The results summarized in FIG. 2 demonstrate that BRAF RNA levels were significantly greater than PIK3CA levels in the sample, and that the relative concentrations of DNA:RNA species varies between subjects.

### Example 2—Whole Genome Sequencing with Libraries Prepared with a RT Step

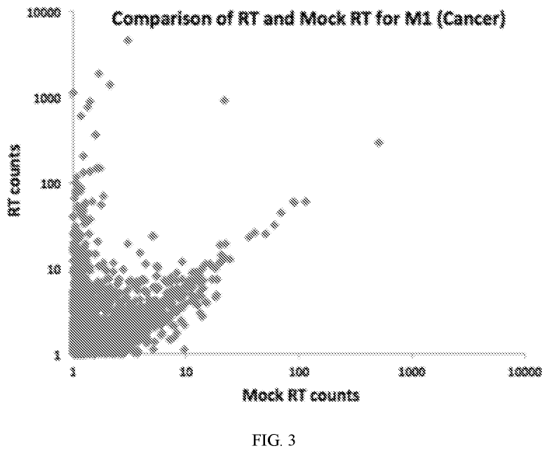

Nucleic acid libraries were prepared from a cell-free sample of nucleic acids including DNA and RNA, with and without a reverse transcription step. The libraries were prepared using a Truseq RNA Access library kit (Illumina, San Diego, Calif.), without performing enrichment. Libraries were sequenced, and sequences were aligned to a total transcriptome. FIG. 3 demonstrates that the number of sequences that aligned with known genes was significantly greater for sequences from the library prepared with a reverse transcription step (RT sequences) than for sequences from the library prepared without a reverse transcription step (mock RT sequences). In addition, the number sequences that aligned with exons, such as exons 4 and 5 of the GNAQ gene and exons of the LINC00152 non-coding gene, was significantly greater for RT sequences than mock RT sequences (data not shown).

### Example 3—Targeted Sequencing with Libraries Prepared with a RT Step

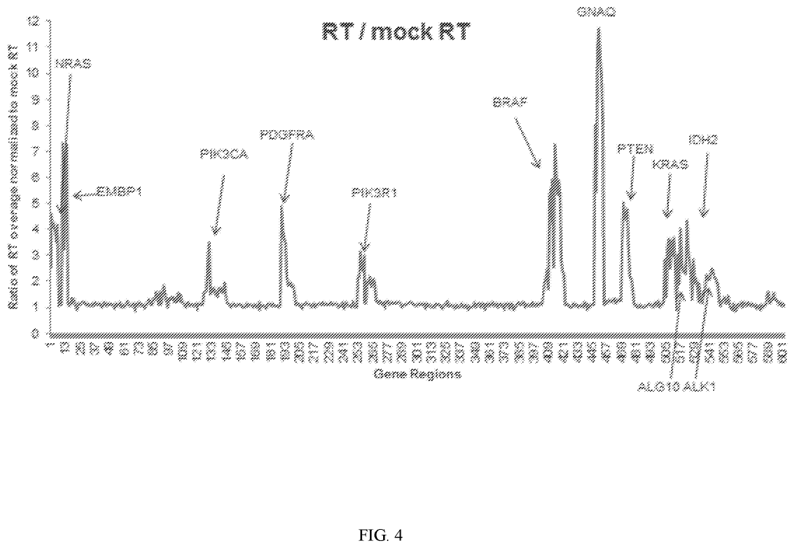

Nucleic acid libraries were prepared from a cell-free sample of nucleic acids including DNA and RNA from a cancer patient, with and without a reverse transcription step. The libraries were prepared using Truseq RNA Access library kit (Illumina, San Diego, Calif.) and enriched using probes designed from a non-small cell lung cancer (NSCLC) V1 panel. Sequences were aligned to targeted genes included in the NSCLC V1 panel. FIG. 4 is a graph of the ratio of coverage for libraries prepared by a method with a reverse transcription step (RT) vs. a method without a reverse transcription step (mock RT), for certain gene regions tested in the NSCLC V1 panel. FIG. 4 shows that coverage for at least 12 genes in the NSCLC V1 panel was more than double for RT sequences than mock RT sequences. The sensitivity of detection of at least 12 genes increased significantly when a reverse transcription was included in library preparation.

The sequencing data was analyzed further for a BRAF gene variant, and a CD44-FGFR2 gene fusion variant. The results of the analysis for each variant are summarized in TABLE 1 and TABLE 2, respectively. For both variants, the sensitivity of detection was significantly increased for RT sequences analyzed from a library that was prepared with a reverse transcription step, compared to mock RT sequences analyzed from a library that was prepared without a reverse transcription step.

TABLE 1

| Sample | Collapsed depth | Number of mutants | Frequency |
|---|---|---|---|
| Mock RT | 1810 | 1 | 0.06% |
| RT | 10894 | 7 | 0.06% |

TABLE 2

| Sample | CD44-FGFR2 fusion frequency |
|---|---|
| Mock RT | 0% |
| RT | 0.2% |

### Example 4—Mutations Detected in Only Libraries Prepared with a RT Step

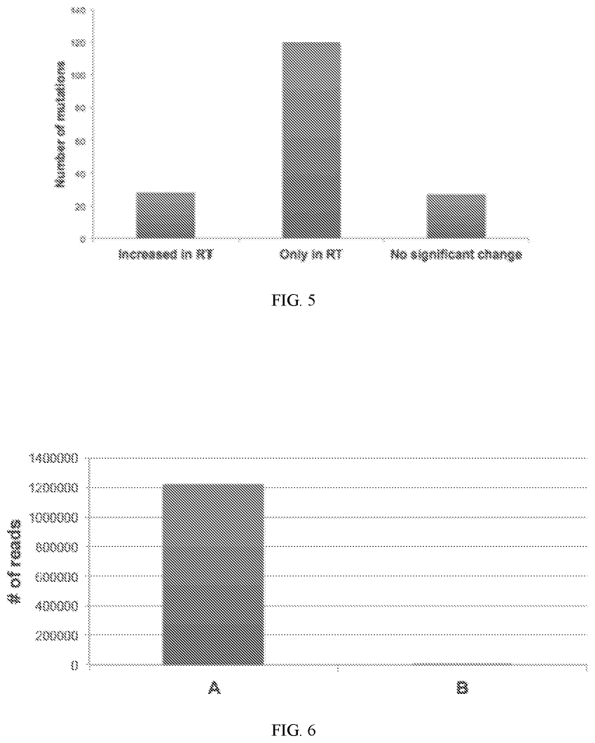

Nucleic acid libraries were prepared from a cell-free sample of nucleic acids including DNA and RNA from 15 cancer patients, with and without a reverse transcription step. The libraries were prepared using Truseq RNA Access library kit (Illumina, San Diego, Calif.) and enriched using probes designed from an NSCLC V1 panel. The libraries were sequenced by targeted sequencing, and sequences were aligned to targeted gene panels. FIG. 5 is a graph of the number of mutations that were found with an increased frequency in the library prepared with a reverse transcription step.

### Example 5—Preparation of a Library in which cDNA Derived from RNA Only was Tagged

Nucleic acid libraries were prepared from a cell-free sample of nucleic acids including DNA and RNA, in the presence of tagged random hexamers, and in the presence or absence of a reverse transcriptase. The libraries were prepared using Truseq RNA Access library kit (Illumina, San Diego, Calif.) and enriched using probes designed from an NSCLC V1 panel. Libraries were sequenced, and the number of reads for tagged sequences was determined for each library. FIG. 6 is a graph of the number of reads from a library prepared with tagged random hexamers either with reverse transcriptase (A); or without reverse transcriptase (B). FIG. 6 illustrates that the tagged sequences were present in the library prepared with reverse transcriptase, and an insubstantial background level of tagged sequences was detected in the library prepared without reverse transcriptase. This demonstrates that sequences of cDNA derived from RNA can be readily identified using tags, and can be distinguished from non-tagged sequences.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1.-43. (canceled)

44. A method for preparing a library of nucleic acids comprising:
(a) hybridizing a plurality of polynucleotides with a plurality of primers, wherein the plurality of polynucleotides comprises RNA and DNA;
(b) extending the hybridized primers with a reverse transcriptase; and
(c) generating a library of nucleic acids from the extended primers and the DNA.

45. The method of claim 44, further comprising (d) sequencing the library of nucleic acids.

46. The method of claim 44, wherein the plurality of primers comprise tags.

47. The method of claim 46, further comprising (e) identifying polynucleotide sequences comprising the tags, thereby identifying sequences derived from the RNA polynucleotides of the plurality of polynucleotides.

48. The method of claim 47, further comprising identifying polynucleotide sequences lacking the tags, thereby identifying sequences derived from the DNA polynucleotides of the plurality of polynucleotides.

49. The method of claim 44, wherein the plurality of primers comprises different sequences.

50. The method of claim 44, wherein the plurality of primers comprises greater than 10,000 different sequences.

51. The method of claim 46, wherein the plurality of primers comprises the same tag.

52. The method of claim 44, wherein the reverse transcriptase lacks a DNA-dependent polymerase activity.

53. The method of claim 44, wherein (b) is performed in the presence of the DNA polynucleotides.

54. The method of claim 44, wherein (b) comprises generating double-stranded cDNA from the extended primers.

55. The method of claim 44, wherein the plurality of polynucleotides is cell-free.

56. The method of claim 55, wherein the plurality of polynucleotides is obtained from a sample selected from the group consisting of serum, interstitial fluid, lymph, cerebrospinal fluid, sputum, urine, milk, sweat, and tears.

57. A method of identifying a nucleic acid in a sample of nucleic acids, comprising:
(i) obtaining sequence data from a library of nucleic acids prepared from a sample of nucleic acids by the method of claim 46; and
(ii) identifying a polynucleotide sequence comprising a tag, thereby identifying a sequence derived from a RNA polynucleotide of the plurality of polynucleotides.

58. The method of claim 57, further comprising identifying a variant in the polynucleotide sequence comprising a tag.

59. The method of claim 58, wherein the variant is selected from the group consisting of a single nucleotide polymorphism (SNP), a deletion, an insertion, a substitution, a duplication, a translocation, and a gene fusion.

60. The method of claim 57, further comprising identifying a reverse transcription error in the polynucleotide sequence comprising a tag.

61. The method of claim 57, further comprising comparing the polynucleotide sequence comprising a tag with a reference sequence derived from a DNA polynucleotide of the library of nucleic acids.

62. The method of claim 57, wherein the RNA polynucleotide is an RNA selected from the group consisting of non-coding RNA, piRNA, siRNA, lncRNA, shRNA, snRNA, miRNA, snoRNA, viral RNA, bacterial RNA, and a ribozyme.

63. A kit for preparing a library of nucleic acids comprising:
a reverse transcriptase lacking a DNA-dependent polymerase activity;
a plurality of primers comprising tags, wherein each primer comprises the same tag, and each primer is different; and
a component selected from the group consisting of a kinase, an RNase, a ligase, a transposon, a polymerase, and a sequencing adaptor.
